(19) United States
(12) Patent Application Publication
(10) Pub. No.: US 2004/0136938 A1
(43) Pub. Date: Jul. 15, 2004

(54) Composition of vitamin c and/or vitamin a

(76) Inventors: Robert Ladislas, Santeny (FR); Alexandre Michel Robert, Santeny (FR); Catherine Sylvie Robert, Paris (FR); Jean-Luc Gesztesi, Sao Paulo (BR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/398,968

(22) PCT Filed: Sep. 11, 2001
(86) PCT No.: PCT/BR01/00115

(30) Foreign Application Priority Data
Sep. 11, 2000 (FR) 00/11546
Mar. 13, 2001 (BR) PI 0100957-5

(51) Int. Cl. A61K 31/715; A61K 7/06; A61K 7/11; A61K 31/375

(52) U.S. Cl. 424/70.13; 514/54; 514/474; 514/559; 514/725

(57) ABSTRACT

The present invention relates to a new cosmetic or pharmaceutical composition charactrized in that it comprises at least on vitamin component chosen from the group consisting of vitamin C and its derivatives, vitamin A (or retinol) and its derivatives, and mixtures of these components, in association with at least one fucose component chosen form the group consisting of fucose, polysacharides and oligosaccharides that contain fucose, sisting of fucose, polysaccharides and oligosaccharides that contain fucose, and mixtures of these components, as well as at least one acceptable excipient. This composition enables one to reduces significantly, by means of a real synergy effect, the toxic effect of the vitamin component and, therefore, to use in the composition contents of the vitamin component higher or equal to the contents of the products that already exist on the market without any risk for the user.

The present invention relates to a new cosmetic or pharmaceutical composition that comprises an association of vitamin C and/or vitamin A with a fucose component, and to the use of this association specially in products of topical application, for which an activity on the epithelial or conjunctive tissue is sought, especially anti-aging products such as pharmaceutical or veterinary products and, more specially, in cosmetic products.

Collagen, the largest constituent of the dermis, undergoes, according to earlier papers (BRANCHET et al, Arch. Gerontol. Geriatr., 1991, 13:1-14), a quantitative decrease with the aging. The regulation of its biosynthesis is therefore a very important step to consider in fighting against skin aging.

If it is well known that ascorbic acid (or “vitamin C”) and its salts (specially sodium) have stimulating effects on the biosynthesis of collagen, on the other hand it has been possible to point out its cytotoxic effect starting from a concentration of about 0.01% by weight.

The activation effect of biosynthesis of collagen, which is quite favorable, can be observed in cliches of electronic microscopy, by a great expansion of the vesicles of the rugose endoplasmic reticulum, full of neosynthesized proteins.

The cytotoxic effect, which is unfavorable and can be observed in millimolar concentrations of ascorbate (about 2.5 mM), manifests itself by displacing the cells of its substrate, by decelerating their proliferation and the cellular feasibility, and then by cellular death.

On the other hand, retinol (or “vitamin A”), as well as other retinoids such as retinyl palmitate, are compounds appreciated especially in the domain of cosmetic products for its biologic activities that are favorable mainly in fighting against skin aging. These activities disclosed by topical utilization of vitamin A and its derivatives are appreciated, for instance, in the article by Wade Cheng, PhD and Shirley De Petris, “Vitamin A Complex”, Skin Inc, March/April, 1998.

However, the use of pure vitamin A or one of its derivatives has the drawback of so high toxicity, that one should limit the dosage or adding components, specially to minimize the discomfort of irritation of the skin that results therefrom.

It was specially pointed out that retinol introduces inhibition of cellular proliferation in fibroblasts in conventional culture (Lacroix A , Anderson G. D. L., Lippman M. E., “Refinoids and cultured human fibroblasts”, Exp Cell Res, 1980, 130: 339-344; Harper R. A., Burgoon T., “Differential effects of retinoic acid on the growth of normal fibroblast-like cells in vitro from, human, swine and rabbit skin”, Cell Biol Int Rep, 1982, 6: 163-170; or else Stumpenhausen G., Hein R., Kulozik M., Mauch C., Bryce G. F., Oono T., Kieg Th., “The influence of retinoids on fibroblasts functions”, inSaurat JH ed., Retinoids: 10 years On, 1991, pp. 139-150 Karger, Basel). This is a real toxic effect of retinol on fibroblasts.

Vitamins C and A and the salts and derivatives thereof being vitamin components often present mainly in cosmetic products, especially anti-aging cosmetic products, it was therefore quite desirable to overcome the above-cited drawbacks, so as to increase the contents and, consequently, the favorable effects of these components, while reducing their toxic effects at the same time.

It has now been found, in an absolutely surprising and unexpected way, that the association of fucose or a polysaccharide or oligosaccharide containing fucose with vitamin C and/or vitamin A enables one to reduce significantly the toxic effects of these vitamins, with a real synergistic effect.

Thus, the present invention has the objective of providing a cosmetic or pharmaceutical composition characterized by comprising at least one vitamin component chosen from the group consisting of vitamin C and its derivatives, vitamin A (or retinol) and its derivatives, and mixtures thereof, in association with at least one fucose component chosen from the group consisting of fucose, polysaccharides and oligosaccharides containing fucose, and mixtures of these components, as well as at least one cosmetically or pharmaceutically acceptable excipient.

By “derivatives of vitamin C” one understands, especially according to the invention, salts such as sodium ascorbate and esters such as ascorbyl phosphate or ascorbyl palmitate.

The term “retinol” (or vitamin A) should be understood as including hydrogenated and non-hydrogenated isomers such as 9-cis-retinol and didehydroretinol.

By “derivatives of vitamin A” one understands, especially according to the invention, other retinolds than retinol, especially esters obtained with retinol and acetic acid, propionic acid, palmitic acid or stearic acid and, more specially, retinoic acid, retinaldehyde (or retinal) and retinyl palmitate.

The term “retinaldehyde” should be understood as including the 4 stereoisomeric forms trans, 13-cis, 11-cis and 9-cis.

The monosaccharide fucose is a deoxyhexose close to galactose, of which it has the stereochemical conformation. However, the structure of fucose essentially differs from the structure of galactose in that the carbon-6 atom has a methyl group (—CH₃) and not a primary alcohol group (—CH₂OH). In fact, this methyl group imparts an interesting partial hydrophobic nature to the molecule of fucose, which is compensated by other hydroxyl groups in the four other carbon atoms present.

The monosaccharide fucose usable in the composition according to the invention may be L-fucose, D-fucose or one of their mixtures. L-fucose and D-fucose may each be in the form of alpha, beta or a mixture of these forms. These products are specially commercialized by SIGMA.

Fucose appears early in the course of phylogenesis, polysaccharides of certain algae and of fungi contain fucose in relatively large quantity, either alone or in combination with other chemical compounds. On the other hand, fucose is widespread in the vegetable kingdom and certain bacteria also synthesize it. Fucose may also appear in the sulfated form, as in fucanes.

Thus, by the expression “polysaccharides and oligosaccharides containing fucose” one understands any polysaccharide or oligosaccharide that comprises at least one fucose unit, including the sulfated polysaccharides and oligosaccharides “fucanes”.

Fucanes are sulfated polysaccharides that form a constituent part of the cellular walls of the stalks of brown algae (Feoficeas). They are also present in certain marine animals such as sea-urchin and sea-cucumber. Raw fucane, also called fucoidanes, obtained by acidic extraction from the cellular walls of the stalks of brown algae, is constituted by a heterogeneous population of molecules, which comprises mainly polymers of sulfated L-fucose of high average molar mass (from 100,000 to 800,000 g/mol). Preparations of fucanes of lower average molar mass (lower than 20,000-10,000 g/mol) have been obtained, for instance, by controlled hydrolysis of fucane of high molar mass, as described in EP-0 403,377, or by radical depolymerization, as described in WO 97/08206. One can also cite especially the product-called “Fucoidane” commercialized by SIGMA.

Among the polysaccharides and oligosaccharides that contain fucose usable in the composition according to the invention other than fucanes, one can cite especially those commercialized by Solabia, such as the polysaccharide “Fucogel 1000”. Application WO 96/23057 describes the process of preparing this polysaccharide, which comprises the repetition motif fucose-galactose-galacturonic acid.

On the other hand, a new mixture of oligosaccharides containing fucones, hereinafter called “Mixture of oligofucoses” specially suitable for the present invention, is found.

This is a mixture of non-sulfated fucose-based oligosaccharides characterized in that it comprises oligosaccharides of less than 13 saccharide units, which comprise at least one fucose unit in a non-reducing end position, and that It can be obtained by means of a process that comprises at least one step of degradation of a polysaccharide from a microorganism of the gender Klebsiella pneumoniae subsp. pneumoniae.

By “non-sulfated fucose-based oligosaccharide” one understands, according to the invention and in accordance with the general knowledge of those skilled in the art, an oligosaccharide that contains at least one unit of saccharide fucose and that has not sulfate group —O(SO₃)⁻. Fucanes are especially excluded from this definition.

By “oligosaccharide that comprise at least one unit of fucose in a non-reducing end position” one understands, according to the invention and in accordance with the general knowledge of those skilled in the art, an oligosaccharide that contains at least one unit of saccharide fucose in an end position of the chain of oligosaccharides, this fucose unit being linked to the next saccharide unit of the rest of the oligosaccharide by an acetal-type linkage.

The numbers of saccharide units may be measured with the help of techniques well known to a person skilled in the art, especially using for this purpose the HPLC chromatography, as described in the examples given below.

Preferably, the Mixture of oligofucoses comprise, based on the total weight of the mixture, at least 15% by weight, and preferably from 20 to 50% by weight of oligosaccharides of less than 13 saccharide units, which comprise at least one fucose unit in a non-reducing end position.

More specially, the Mixture of oligofucoses is characterized by comprising, on the other hand, based on the total weight of the mixture, from 25 to 45% by weight of oligosaccharides that contain from 13 to 24 saccharide unit comprising at least one fucose unit in a non-reducing end position.

Still more specially, the Mixture of oligofucoses is characterized in that it comprises, on the other hand, based on the total weight of the mixture, from 15 to 35% by weight of oligosaccharides of more than 54 saccharide units comprising at least one fucose unit in a non-reducing end position.

Since the Mixture of oligofucoses can be obtained by a process that comprises at least one step of degradation of a polysaccharide from a microorganism of the gender Kiebsiella pneumoniae subsp. pneumoniae, the oligosaccharides preferably comprise, at least in part, the repetition motif fucose-galactose-galacturonic acid.

Especially, the Mixture of oligofucoses is susceptible of being obtained by the process that comprises the steps of

a) causing the microorganism of the gender Kiebsiella pneumoniae subsp. pneumoniae to grow in an aqueous nutritive medium by aerobic fermentation of an assimilable source of glucide;

b) recovering the polysaccharide formed from the fermentation must;

c) subjecting the polysaccharide to a moderate hydrolysis;

d) subjecting the hydrolysis product of the step c) to an enzymatic hydrolysis; and

e) deactivating the enzyme and then recovering the Mixture of oligofucoses thus obtained.

More specially, these steps a) to e) may be described as follows.

Step a)

One uses preferably the microorganism Klebsiella pneumoniae subsp. pneumoniae, which is a microorganism deposited in the National Collection of Cultures of Microorganisms under number 1-1507, or a mutant thereof. On the other hand, this microorganism is described in detail in application WO 96/23057.

The aqueous nutritive medium may be any aqueous medium known to a person skilled in the art, which contains sources of carbon, nitrogen and mineral salts, as described in application WO 96/23057.

One may conduct the fermentation at temperatures on the order of from 25 to 35° C., at a pH of from about 6.0 to 7.5, in conditions of aeration and stirring, during periods of time ranging from 2 to 4 days.

The fermentation may be effected in a classic fermenter, by inoculating previously sterilized nutritive medium, for instance, by heating up to a temperature on the order of 120° C. or by sterilizing filtration.

Step b)

At the end of the period of fermentation, one recovers the fermentation must, from which a fucose-rich polysaccharide is isolated in the following way.

The fermentation must is subjected to a heat treatment at a temperature specially ranging from about 100 to about 130° C., preferably from about 115 to about 125° C., for a period of time ranging from 30 minutes to about 2 hours and, preferably, from about 40 minutes to about 1 hour, and at a pH specially ranging from about 2 to about 5.5 and, preferably, from about 3 to about 5.5.

The product of the heat treatment is filtered according to classic means, such as press filters with plates.

In this way, one obtains a limpid, viscous polysaccharide, free from any cell.

Then one carries out precipitation in an alcohol solvent, preferably an alcohol solvent chosen from ethanol, isopropanol and mixtures thereof. One specially uses from about 1 to about 3.0 volume of solvent to 1 volume of polysaccharide and, preferably, from about 1.3 to about 2.0 volume of solvent to 1 volume of polysaccharide.

Then a drying is carried out under vacuum, at a temperature specially ranging from about 20 to about 60° C. and, preferably, from about 30 to about 50° C., until a powder is obtained.

Steps c) and d): Hydrolysis of the Polysaccharide

This is an essential combination of steps. Indeed, one has found, in a surprising and unexpected way, that the combination of a step of moderate hydrolysis, preferably by irradiation with gamma rays and/or by protolysis, with an enzymatic hydrolysis step, enables one to obtain advantageously a sufficient global output of hydrolysis, close to that of a classic hydrolysis, such as an acidic hydrolysis, but with the advantage of specific cuts of an enzymatic hydrolysis. In particular, an acidic classical hydrolysis does not enable one to obtain the mixture of specific oligofucoses, as it leads to the obtention of statistic, redhibitory cuts, as to the random nature. On the other hand, the compounds resulting from a classic acidic hydrolysis prove to be biologically inactive.

Step c): Moderate Hydrolysis of the Polysaccharide

Moderate hydrolysis is carried out by a treatment with gamma rays, a protolysis treatment or by these two successive treatments. Preferably, one successively carries out a treatment with gamma rays and then a protolysis treatment.

The treatment with gamma rays proved to cause a sensible drop in viscosity by a limited degradation, attributable to the action of free radicals. It may be carried out with irradiation means known to those skilled in the art.

This treatment by gamma rays, which are very penetrating rays, presents, in addition, the advantage of sterilizing the polisaccharide, killing the germs present, which could induce inflammation or even cause granuloma. In this way, one prevents a bacterial attach, without having to add to the medium any antiseptic products that could interfere in an undesirable way with the biologic activities of the end product.

The polysaccharide powder obtained in step b), possibly irradiated with gamma rays, may therefore, equally, be subjected to a protolysis treatment. For this purpose, it is placed in an aqueous solution, specially at the proportion of from 1 to 20% by weight and, preferably, from 2 to 10% by weight, with respect to the total weight of the aqueous solution.

The aqueous solution is subjected to a heat treatment, that is to say, a heating up to a temperature specially ranging from about 75 to about 120° C. and, preferably, from about 90 to about 100° C., for a period of time ranging from 1 to 6 hours, in the presence of a proton-generating resin, such as those commercialized and well known to a person skilled in the art, that is to say, a resin generating protons that bring about a cut of the glycosidic linkages with fixation of a water molecule.

Step d): Enzymatic Hydrolysis

One introduces an acidic buffer such as a citric acidic buffer (4.15 g/kg) disodium hydrogenophosphate (about 10.75 g/kg) in the hydrolysate obtained in step b). One regulates the temperature of the solution specially to a temperature ranging from about 25 to about 45° C. and, preferably form about 30 to about 40° C.

One introduces an enzymatic preparation comprising at least one endofucosidase preferably Ferrnizyme HCP such as commercialized by Gist Brocades, according to contents specially from about 2 to about 20% by weight and, preferably, from about 5 to about 15% by weight, with respect to the initial weight of polysaccharide powder utilized.

The thus obtained mixture is maintained under stirring for a period of time ranging from about 8 to about 24 hours and, preferably, from about 10 to about 20 hours, at a temperature specially ranging from about 25 to about 45° C. and, preferably, from about 30 to about 40° C., the pH being regulated at 6 by the presence of the buffer mixture.

Step e)

The hydrolysis product obtained after the step d) is filtered according to classical means such as a press filter with plates.

The collected solution is then heat-treated at a temperature specially ranging from about 75 to about 120° C. and, preferably, from about 90 to about 105° C., for a period of time specially ranging from about 10 to about 45 minutes and, preferably from about 20 to about 35 minutes, in order to deactivate the enzyme and, more particularly, the fucosidase activity of this specific enzyme.

One lets it cool down to a temperature specially ranging from about 20 to about 40° C.

While it is cooling, preservatives may by added to the solution.

One then filters the whole under sterile conditions, and then the packaging is carried out.

The thus obtained mixture of oligofucoses may be characterized with the aid of techniques well known to those skilled in the art, specially HPCL, chromatography on the thin layer and other methods and chemical dosages.

In this way, one can find out that the oligosaccharides of the mixture are such that fucose is mainly at the end of the chain in a non-reducing end position.

The favorable effects of the composition according to the invention manifest themselves in minor concentrations of fucose components, preferably at a concentration ranging from about 0.001 to about 20% by weight, and more preferably from about 0.01 to 10% by weight, the concentration in vitamin component raging preferably from about 0.001 to about 90% by weight, and more preferably from about 0.01 to about 10% by weight, based on the total weight of the composition.

By choosing adequately the respective concentrations of the vitamin component and of the fucose component, it is possible to reduce significantly, and even to suppress the toxic effect of the vitamin component and, therefore, to use, in the composition according to the invention, contents of vitamin component higher or equal to the contents of product that already exist in the commerce, without any risk for the user.

Preferably, the weight ratio of vitamin component:fucose component ranges from about 800:1 to about 1:2, and more preferably from about 600:1 to about 1:1.

The cosmetically or pharmaceutically acceptable excipient may be any one from those known to a person skilled in the art for the purpose of obtaining a composition according to the invention in the form of a cream, a lotion, a gel, a salve, etc., possibly in the form of an emulsion, having, in addition, other components known to a person skilled in the art, to improve, modify or stabilize the composition from a cosmetic or pharmaceutical point of view.

The expression “pharmaceutically acceptable excipient” embraces excipients adapted for a veterinary use of the composition, according to the invention.

The composition according to the invention may, in particular, contain other additives and aids to the formulation, such as antioxidant agents for fighting free radicals. One can cite specially pure vitamin E or di-alpha-tocopherol and its derivatives, and 2,6-di-tert-butyl-p-cresol (BHT).

According to a special mode of carrying out the present invention, the composition comprises, on the other hand, a vector, such as microspheres that contain especially the vitamin component, as for example, the “Talaspheres” described in U.S. Pat No. 5,395,620 or in patent application PI 9706994-7 of the same applicant.

The composition according to the invention may comprise, for instance, a plurality of dispersed microspheres, which comprise a first vitamin-C component in a first group of microspheres and a second vitamin-A component in a second group of microspheres, the fucose component being outside the microspheres, in the rest of the composition. Naturally, a variant of this mode of carrying out the invention may consist in that the vitamin-C and/or vitamin-A component is comprised in a single group of microspheres.

Advantageously, the composition according to the invention may further comprise, in particular, at least one cosmetically or pharmaceutically acceptable additive chosen from the group consisting of the agents structuring the skin (such as squalane and sphingolipides), the moistening agents (such as glycerin and hydroxy prosilan C), the emollients (such as butylene glycol and cetyl lactate, the silicones (such as cyclomethicone), the sun protection agents (such as Parsol 1789 and Eusolex 6300), the emulsifiers (specially Carbopol 1342 associated to triethanolamine and soybean lecithin), the thickeners (notably xanthan gum), the scavengers (specially EDTA), the antioxidants (such as BHT described above), the fragrances, the preservatives and mixtures thereof.

Of course, the operational conditions for preparing the composition according to the invention are part of the general knowledge of those skilled in the art.

The present invention has further the objective of using, in a cosmetic or pharmaceutical composition, of at least one vitamin component such as defined above, in association with at least one fucose component as defined above, to reduce the toxic effects of the vitamin component.

Preferably, one uses a weight ratio of vitamin component:fucose component ranging from about 800:1 to about 1:2, and more preferably from about 600:1 to about 1:1.

Finally, the present invention further has the objective of providing a method for cosmetic or pharmaceutical treatment of the skin, characterized in that one applies to the skin a cosmetic or pharmaceutical composition as defined above.

Especially, the present invention has the objective of providing a cosmetic treatment of the skin, characterized in that one applies to the skin a cosmetic composition as described above.

The examples given below illustrate a real synergy between fucose, the oligosaccharides with fucose with the retinol and the ascorbate, and justify their association specially in a cosmetological “anti-aging” preparation.

The following examples, however, are only intended to illustrate the present invention and should not at all be taken as limiting the scope of the invention.

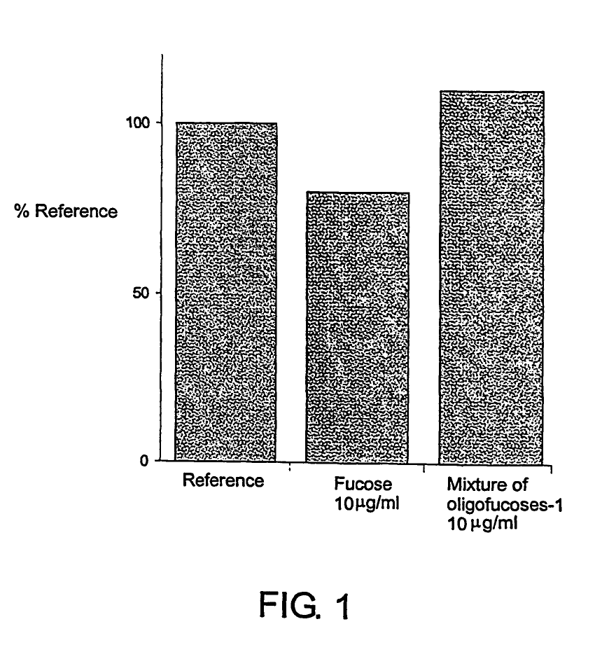

FIG. 1 is a histogram that brings the results presented in example 4.b.1, in terms of percentage of effectiveness of fucose and of the Mixture of oligofucoses-1 for stimulating the synthesis of collagen by the fibroblasts.

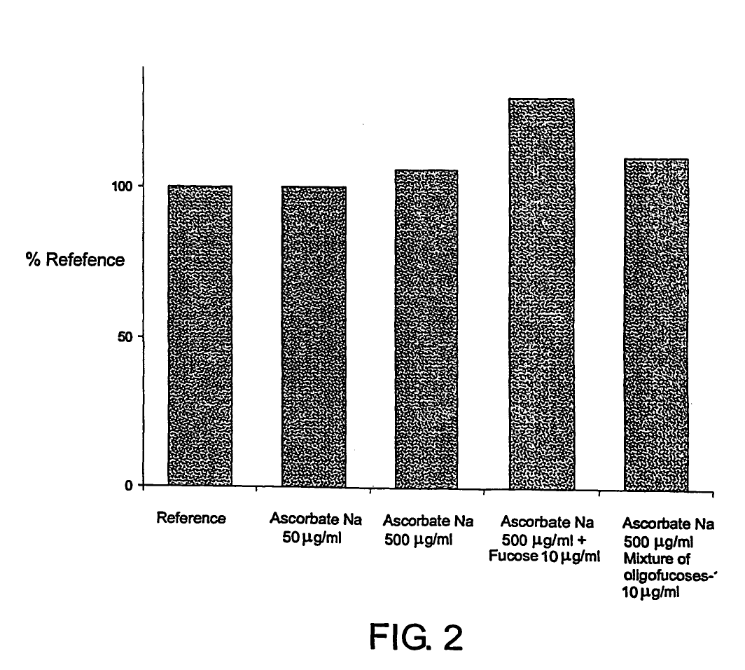

FIG. 2 is a histogram that brings the results presented in example 4.b.2, in terms of percentage of effectiveness of fucose and of the Mixture of oligofucoses-1 for stimulating the biosynthesis of collagen by the fibroblasts in the presence of sodium ascorbate.

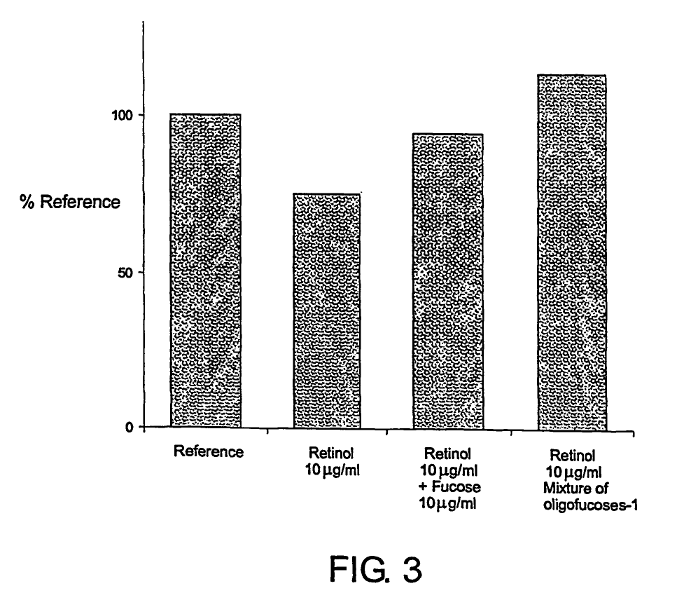

FIG. 3 is a histogram that brings the results presented in example 4.b.3, in terms of percentage of effectiveness of fucose and of the Mixture of oligofucoses-1 for stimulating the synthesis of collagen in the presence of retinol.

### EXAMPLE 1

### Preparation of a Mixture of Oligofucoses

a) Fermentation

One uses the microorganism Klebsiella pneumoniaesubsp. pneumoniae, which is a microorganism deposited in the National Collection of Microorganisms under No. 1-15097. The nutritive medium and other conditions of the fermentation are as follows.

Preparation of the inoculums

Culture medium:

Neosorb® 70-07 (sorbitol contents: 70% M.S.;

sold by ROQUETTE FRERES, Lille/France): 17.90 g/l (that is, 12.5 g/l of sorbitol)

Peptone Biokar 104003 (protein hydrolisate,

sold by SOLALBIA-BIOKAR, Pantin, France): 4.50 g/l

Yeast extract: 0.05 g/l

KH₂PO₄: 1.50 g/l

K₂HP0₄: 4.50 g/l

MgSO₄7H₂O: 0.20 g/l

Pluronic® PE 61000 (antifoaming agent,

sold by BASF, D-6700 Ludwigshafen, Alemanha): 0.50 g/l

Placing in solution into water

Culture condition:

Sterilization at 121° C. for 30 minutes

Culture temperature: 30° C.

Inoculation rate: 5-10%

Aeration: 1 VVM

pH not regulated (pH of about 7.00)

Duration of culture: 24 hours

Production medium

Culture medium:

Neosorb® 70-07: 54.00 g/l (that is, 38 g/l of sorbitol)

Peptone Biokar 104003: 4.50 gl

Yeast extract: 0.05 g/l

KH₂PO₄: 1.50 g/l

MgSO₄, 7H₂O: 0.20 g/l

Pluronic® PE 61000: 0.50 g/l

Placing in solution into water

Culture conditions (fermenter Chemap, which has a useful volume of 350 liters):

Sterilization at 120° C. for 45 minutes

Culture temperature: 30° C.

Inoculation rate: about 5%

Stirring: 300 rpm (Rushton-type stirrer)

Aeration: 1 VVM

pH regulated at 7.0 by NaOH 7N

Pressure: 100-200 mbars

Duration of culture: 60-65 hours

Average values achieved in production:

Viscosity at the end of the cycle: 40000 MPa.s (Viscosimeters Brookfield DV-II+model LV, movable body SP 31, chamber SC4-34/13R, 30° C.)

Concentration of the polysaccharide produced in the medium, calculated in L-fucose: 2 g/l (Methods: Dische and Shettles)

Sorbitol consumed: >35 g/l (in sorbitol)

NaOH at 20% by weight consumed: 15 liters/M³

Start of regulation of the pH: 16-17 hours after inoculation of the fermenter

Final dry extract of the fermentation medium: ˜20 g/l

b) Recovery of the Formed Polysaccharide

One subjects the fermentation must to a heat treatment at a temperature of 120° C. for 45 minutes and with a pH of 5.5. The product of the heat treatment is filtered with the help of a press filter with Seitz-type plates. In this way, one obtains a limpid, viscous polysaccharide, free from any cell. Then one carries out a precipitation in 1.5 volume of ethanol to 1 volume of polysaccharide. Then a drying is carried out under vacuum at a temperature of 25° C. until a powder is obtained. Considering the microorganism used, this polysaccharide is composed of repetitive trisaccharide units of fucose—galactose—galacturonic acid, and thus it presents the following structure:

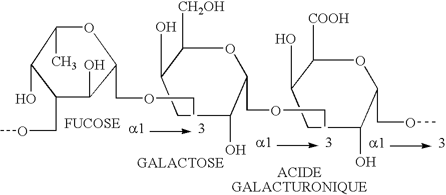

c) Moderate Hydrolysis of the Polysaccharide

The polysaccharide powder is placed in aqueous solution at the proportion of 5% by weight, based on the total weight of the aqueous solution. The aqueous solution is subjected to a heat treatment, that is to say, heating up to 100° C., for 3 hours, in the presence of a proton-generating resin.

d) Enzymatic Hydrolysis

One introduces the buffer mixture of citric acid (4.15 g/kg)-disodium hydrogenphosphate (about 10.75 g/kg) into the hydrolysate. The temperature of the solution is regulated at 37° C. One introduces the enzymatic preparation Fermizyme HCP, as commercialized by Gist Brocades, according to contents of 10% by weight, based on the initial weight of polysaccharide used, that is to say, 0.05% by weight with respect to the total mass of the aqueous solution after placing the powder into water, as described above for the moderate hydrolysis by protolysis.

The thus obtained mixture is kept under stirring for 15 hours at a temperature of 37° C., the pH being regulated at 6 by the presence of the buffer mixture.

e) Deactivation of the Enzyme and Recovery of the Mixture of Oligosaccharides

The product of hydrolysis is filtered with a press filter with Seitz-type plates. The solution collected is then heat-treated at 100° C., for 30 minutes, to deactivate the enzyme. One lets it cool at a temperature of 25° C. When it is cooling, the preservatives phenoxy ethanol (1% by weight) and phenonipe (0.3% by weight) are added to the solution. Then the whole is filtered in sterile conditions.

The thus obtained mixture of oligosaccharides is called “Mixture of oligofucoses-1”.

### EXAMPLE 2

### HPLC Characterization of the Mixture of Oligofucoses-1

The fractionation of the Mixture of oligofucoses-1 obtained in Example 1 was carried out with the objective of determining the proportion of oligo- and of polysaccharides in its composition.

a) Fractionation by Preparative Exclusion Chromatography on Column “XK 50/60 Superdex 75 Prepgrade”

One passes 50 ml of the Mixture oligofucoses-1 concentrated at 50 mg/ml, on a preparative column “XK 50/60 Superdex 75 prepgrade” (exclusion chromatography) and collects 95 fractions, then passed in HPLC.

TABLE 1
Technical informations referring to the preparative
column XK 50/60 Superdex 75 prepgrade

| | |
|---|---|
| Packing | Superdex 75 prepgrade (34 μm) |
| Column size | Height: 50 cm |
| | Diameter: 60 mm |
| Column type | XK 50/60 |
| Usable interval of fractionation | 5 × 10² Da-3 × 10⁴Da |
| Injected sample | 50 ml of the mixture of oligofucoses-1 |
| | at concentration of 50 mg/ml (total: |
| | 2.50 g) |
| Elution Speed | 1 ml/minute |
| Number of collected fractions | 95 fractions of 12.5 ml each |
| Movable phase | PBS |

After fractionation the Mixture of oligofucoses-1 on the column “XK 50/60 Superdex 75 prepgrade”, 95 fractions are collected, 45 of which contain osides.

b) Characterization of the Fractions Obtained by HPLC (Exclusion Chromatography), Ultrahydrogel 120 and Ultrahydrogel 250 Columns

The objective of this second part of the study was to pass all the 95 fractions of the mixture of oligofucoses-1 on a HPLC exclusion column (Ultrahydrogel 120 and 250 columns), in order to analyze the molecular weights and the concentration of the components of these fractions.

For this study one has worked with a Waters HPLC system, the description of which follows.

TABLE 2
technical characteristics of the HPLC chromatography
system used.

| | |
|---|---|
| Apparatus | HPLC Waters 600 |
| Columns | Ultrahydrogel 120 (pore size: 120 Å) and Ultrahydrogel |
| | 250 (pore size: 250 Å) from Waters. Size: 7.8 mm × |
| | 300 mm, containing the gel of hydroxylated |
| | polymethacrylate |
| Injected | 20 μl per automatic injector |
| samples | |
| Elution | 0.10 M NaNO₃ |
| Time of elution | 50 minutes/samples |
| Elution speed | 0.5 ml/minute |
| Detection | By measuring the refraction index with a Waters |
| | 410 refractometer |

TABLE 3
standards of molecular weights of polyethylene
glycol (Fluka) used for the HPLC exclusion chromatography

| Molecular weight | Time of elution (minutes) |
|---|---|
| 400 | 37.892 |
| 600 | 36.026 |
| 1000 | 33.940 |
| 2000 | 31.154 |
| 4000 | 28.896 |
| 6000 | 27.868 |
| 8000 | 26.946 |
| 12000 | 26.192 |
| 20000 | 25.308 |
| 35000 | 24.216 |

c) Results

The molecular weights of the components of the studied fractions were calculated, by using the following equation, obtained with the standards of molecular weights of Fluka, described in Table 3:

Molecular weight of the components=55290000 *10\hat{ }(-13942* x)

R²=0.982

X=time of elution (minutes)

The first fraction containing components of the Mixture of oligofucoses-1 is fraction No. 44 and the last one is fraction No. 89, which means that the same component of the less elevated molecular weight is obtained after 89 fractions collected (after an elution of 89×12.5 ml=1112.5 ml). The fractions collected contain mono-, oligo- and polysaccharides of 184 Da (mixture of monosaccharides) up to about 21 kDa. Therefore, this fraction contains polysaccharides formed by an average of 117 monosaccharide units or of 39 trisaccharide units.

Most of the fractions, with the exception of fractions No. 77, 78, 79, 81, 82, 83, 84, 85, and 86, contain a single saccharide peak (separation limited by the sensitivity of the separation method applied).

The approximate concentration of the different fractions may be determined by using an appraisal range of fucose standard at growing concentrations. This kind of “mono-compositional” standard range could be used thanks to the detection system (measure of the refraction index with a refractometer). According to these results, a solution of 1 μg/ml of fucose gives, on an average, a surface peak of 29409 (arbitrary units of the system). Knowing the surfaces of the peaks analyzed, it was possible to calculate their apparent concentrations.

The achieved results show that the Mixture of oligofucoses-1 contains approximately 26% of small osides (up to 2 kDa, about 4 trisaccharide units), about 36% of oligosaccharides (up to 4 kDa, 8 trisacharide units) and about 23% of polysaccharides of molecular weight higher than 10 kDa (18 trisaccharide units).

Taking into consideration the microorganism and the specific enzyme (endo-fucosidase) used for preparing the Mixture of oligofucose-1, it follows that the oligosaccharides of the mixture contain a fucose unit in non-reducing end position.

### EXAMPLE 3

### Action of the Association of the Mixture of Oligofucoses-1 with Sodium Ascorbate and/or Retinol on the Fibroblasts of Human Skin

The Mixture of oligofucoses-1 as prepared in example 1 is tested in concentrations of 1 μg/ml and 10μg/ml, both alone and in the simultaneous presence of 375 μg/ml of sodium ascorbate and/or 20 μg/ml of retinol.

a) Methodology

a .1) Study of the Cellular Proliferation

The fibroblasts of human skin used in this study come from the removal of skin from a 20 years old woman (28^{th} passing). The cells were cultivated on 12-well plates, in a DMEM culture medium with 10% of fetal calf serum (SVF), 1% of antibiotics and of antifungus (PSF), and 1 μCi/ml of [³H]-timidine (ICN) for 72 hours in the presence of the products to be tested at final concentrations of 1 μg/ml and 10 μg/ml.

After 72 hours of culture in stove (5% (v/v) CO₂, 95% (v/v) air) at 37° C. in the presence of the tested products, the cells were washed four times with PBS, then the cellular carpet was detached for 0.05% of tripsin. Three ml of scintillation liquid were then added per sample, then the radioactivity incorporated in the cells is read in a computer with scintillation.

a .2.) Synergy with Retinol

One incubated the cells with 20 μg/ml of retinol (69.72 μM). Retinol induces, in the absence of the oligosaccharides, a decrease in the cellular proliferation of about 45% (table 2 below). Two concentrations were tested: 1 μg/ml and 10 μg/ml.

a .3) Study of Synergy with Na Ascorbate

One incubated the cells with 375 μg/ml of Na ascorbate (1.875 mM). Vitamin C induces, in the absence of fucose or of the Mixture of oligofucoses-1, a decrease in the cellular proliferation of about 60% (table 3 below). Two concentrations were tested: 1 μg/ml and 10 μg/ml.

a .4) Synergy with Na Ascorbate and Retinol

The cells were simultaneously incubated with 375 μg/ml of Na ascorbate and 20 μg/ml of retinol. This combination produces a great inhibition of the proliferation, partly obtained by the tested products.

b.) Results

b.1) Action on the Cellular Proliferation

72-hour incubation of the fibroblasts with the Mixture of oligofocoses-1 stimulated the cellular proliferation in comparison with the non-treated cells (table 1 below).

b.2) Synergy with Retinol

72-hour incubation of the fibroblast with 20 μg/ml of retinol caused a decrease of 42.5% in the cellular proliferation (table 2 below) with respect to the reference without retinol.

In the presence of 20 μg/ml of retinol and of different concentrations of the tested products, simultaneously added to the culture media of the fibroblasts, the cellular proliferation is significantly higher than in the presence of 20 μg/ml of retinol alone. This is a protecting effect that indicates synergy between retinol and the Mixture of oligofucoses-1.

1 μg/ml and 10 μg/ml of the Mixture of oligofucoses-1 increased significantly the cellular proliferation (+24% and +27%, respectively) with respect to the reference (20 μg/ml of retinol alone, table 2 below).

b.3) Synergy with Na Ascorbate

72-hour incubation of the fibroblasts with 375 μg/ml (1.875 mM) of Na ascorbate decreased the incorporation of [³H]-timidin, and therefore the cellular proliferation, by about 36% with respect to the control (table 3 below).

In the simultaneous presence of 375 μg/ml of Na ascorbate and of different concentrations of the tested product, the cellular proliferation of the fibroblasts increased significantly with respect to ascorbate alone (table 3 below). The Mixture of oligofucoses-1 is effective both at 1 μg/ml and 10 μg/ml.

b.4) Synergy with Ascorbate and Retinol

In the simultaneous presence of 375 μg/ml of Na ascorbate and of 20 μg/ml of retinol, the cellular proliferation of the fibroblasts decreased by about 88% with respect to ascorbate alone (table 4 below).

TABLE 4
EFFECT OF THE DIFFERENT CONCENTRATIONS
OF THE MIXTURE OF OLIGOFUCOSES-1 ON
THE CELLULAR PROLIFERATION OF THE
FIBROBLASTS OF HUMAN SKIN (PROLIFERATION
WITH RESPECT TO THE CONTROL)

| | | | Statistic |
|---|---|---|---|
| | | Effectiveness | meaning |
| | | with respect | (p with |
| | | to the | respect to |
| Product | Concentration | control | the control) |
| Control | | | |
| Mixture of oligofucoses-1 | 1 μg/ml | +4.4 | N.S. |
| Mixture of oligofucoses-1 | 10 μg/ml | +20.6 | *0.019 |

TABLE 5
Effect of the different concentrations of the Mixture
of oligoficoses-1 on the cytotoxicity of 20 μg/ml of retinol
(proliferation with respect to the control and with respect
to 20 μg/ml of retinol)

| | | | | Statistic | Statistic |
|---|---|---|---|---|---|
| | | % 20 μg/ml | % vs. | meaning (p | meaning (P |
| Treatment | concentration | of retinol | control | vs. Retinol) | vs control) |
| Retinol | 20 μg/ml | | −42.5 | | 0.019 |
| Mixture of | 1 μg/ml | +24.2 | −29.3 | *0.047 | 58 |
| oligofucoses-1 + | | | | | |
| ascorbate | | | | | |
| 375 μg/ml | | | | | |
| Mixture of | 10 μg/ml | +26.9 | −27.1 | *0.046 | 54 |
| oligofucoses-1 + | | | | | |
| ascorbate | | | | | |
| 375 μg/ml | | | | | |

TABLE 6
Effect of the different concentrations of the Mixture
of oligofucoses-1 on the cytotoxicity of 375 μg/ml of Na
ascorbate (proliferation with respect to the control and with
respect to the 375 μg/ml of ascorbate)

| | | % vs. 375 μg/ml | | Statistic | Statistic meaning |
|---|---|---|---|---|---|
| | | of | % vs. | meaning (p | (P vs |
| Treatment | concentration | ascorbate | control | vs. ascorbate) | control) |
| Na ascorbate | 375 μg/ml | | −64.4 | | 05 |
| Mixture of | 1 μg/ml | +96.9 | −30.0 | **0.003 | 51 |
| oligofucoses-1 | | | | | |
| Mixture of | 10 μg/ml | +80.8 | −35.7 | *0.015 | 33 |
| oligofucoses-1 + | | | | | |
| ascorbate | | | | | |
| 375 μg/ml | | | | | |

TABLE 7
Effect of the different concentrations of the Mixture
of oligofucoses-1 on the cytotoxicity of 375 μg/ml of Na
ascorbate and of 20 μg/ml of retinol (proliferation with
respect to the control and with respect to 375 μg/ml of
ascorbate + 20 μg/ml of retinol)

| | | % vs. | | | Statistic |
|---|---|---|---|---|---|
| | | Ascorbate + | % vs. | Statistic meaning | meaning (P |
| Treatment | concentration | retinol | control | (p vs. + retinol + ascorbate) | vs control) |
| Na ascorbate | 375 μg/ml | | −64.4 | | 05 |
| Retinol | 20 μg/ml | | −42.5 | | 19 |
| Na ascorbate + | 375 μg/ml + | | −87.54 | | 03 |
| retinol | 20 μg/ml | | | | |
| Mixture of | 1 μg/ml | +95.2 | −75.7 | *0.010 | 04 |
| oligofucoses-1 + | | | | | |
| ascorbate | | | | | |
| 375 μg/ml | | | | | |
| Mixture of | 10 μg/ml | +102.8 | −74.7 | **0.007 | 04 |
| oligofucoses-1 + | | | | | |
| ascorbate | | | | | |
| 375 μg/ml | | | | | |

### EXAMPLE 4

### Effect of Fucose and of the Mixture of Oligofucoses-1 in the Presence of Sodium Ascorbate and/or Retinol.

One studied the influence of fucose and of the Mixture of oligofucoses-1, as prepared in example 1, on the cytotoxic effect of the sodium ascorbate, as well as the influence of these two fucose components on the effect of retinol.

a) Methodology

The fibroblasts of mammaplasty of a 45 years old woman, in passage 14, were seeded on 12-well plates at the rate of 0.5.10⁵ cells per well. The cells are placed in culture for 48 hours in the presence of a DMEM culture medium at 10% of fetal calf serum (SVF), in stove (5% (v/v) CO₂, 95% (v/v) air) at 37° C.

After rinsing with PBS, they are treated and placed again in culture for 48 hours in the presence of products to be tested and of DMEM without SVF.

The method is based on a specific coloration of collagen by the Sirius red. The cells are directly fixed by the Bouin liquid (1 ml/well) for 1 hour, after exhaustive rinsing with PBS. The fixer is then aspirated and the plates are rinsed with running water by immersion for 15 minutes.

The coloration is carried out under stirring for 1 hour (1 ml/well) and the plates are then rinsed with hydrochloric acid 0.01 N. Then the material is dissolved in 200 μl of sodium hydroxide 0.1N before transferring to the microtiter plates (Nunc). The optical density is measured at 550 nm against sodium hydroxide as a blank.

The counting of the cells is carried out in 4 wells of each plate, and one detaches the cells with tripsin at 0.05%.

One studied the effect of fucose and of the Mixture of oligofucoses-1 (at 10 μg/ml each) in the presence or absence of sodium ascorbate at 500 μg/ml. And then in the presence or absence of retinol (10 μg/ml).

b) Results

b.1) Effects of Fucose and of the Mixture of Oligofucoses-1

One found a decrease (about 19%) in the amount of collagen synthesized by the fibroblasts in the presence of fucose. This decrease is much greater with concentrations of 1 μg/ml and 10 μg/ml than with 100 μg/ml. The Mixture of oligofucoses-1 does not have a significant effect at 10 μg/ml. These results are represented in FIG. 1.

b.2) Effects of Fucose and of the Mixture of Oligofucoses-1 in the Presence of Sodium Ascorbate

One found an increase in the amount of collagen synthesized by the fibroblasts. Sodium ascorbate causes not only disappearance of the expected inhibition by fucose, but it even induces stimulation of the biosynthesis of collagen. The same stimulating effect of ascorbate is observed in the presence of the Mixture of oligofucoses-1, but the stimulation observed is lower.

Sodium ascorbate at 500 μg/ml alone activates the synthesis of collagen very little with respect to the effect of 50 μg/ml. By adding fucose, one observes an increase in this stimulation, but modest with the Mixture of oligofucoses-1. These results are represented in FIG. 2.

b.3) Effects of Fucose and of the Mixture of Oligofucoses-1 in the Presence of Retinol

Retinol alone has an inhibiting effect on the synthesis of collagen. In the presence of fucose, this inhibition is greatly abolished; it is not only abolished but, in addition, a slight stimulation in the presence of the Mixture of oligofucoses-1 occurs. These results are represented in FIG. 3.

c) Conclusion

Free fucose exerts a slight inhibition on the biosynthesis of collagen by the fibroblasts in cultures, whereas the Mixture of oligofucoses-1 does not exert this effect.

In the presence of sodium ascorbate at 500 )μg/ml, the inhibition by fucose disappears and one even observes the existence of greater stimulation in the presence of fucose. In the presence of the Mixture of oligofucoses-1, this stimulation is again found, though somewhat more modest.

Retinol alone at 10 μg/ml inhibits the biosynthesis of collagen. This inhibition is completely abolished by adding the Mixture of oligofucoses-1.

These results are represented in FIGS. 1-3.

### EXAMPLE 5

### Cream Against Aging

| Component | % (by weight) |
|---|---|
| Water | q.s.p. 100% |
| Sodium benzoate | 0.2 |
| Disodium EDTA | 0.08 |
| Glycerin | 2.00 |
| Butylene glycol | 4.00 |
| Carbomer and ETD 2020 | 0.20 |
| Ceteareth-20 | 1.00 |
| Mineral oil | 3.00 |
| Squalane | 2.00 |
| Octyl palmitate | 6.00 |
| Karite butter (“Shea Butter”) | 2.50 |
| Cetearyl alcohol | 1.00 |
| Rosa AFFF Rubiginosa seed oil | 0.20 |
| Decyl oleate | 0.50 |
| Octyl methoxycinamate | 5.00 |
| Butyl methoxy-dibenzoylmethane | 0.50 |
| BHA | 0.01 |
| Cyclomethicone | 5.00 |
| Cyclomethicone & Dimethiconol | 2.00 |
| Dimethicone | 2.00 |
| Fragrance (Crematest Feno) | 0.09 |
| Fragrance (Chemoderm) | 0.09 |
| Triethanolamine | 0.30 |
| 2-Bromo-2-Nitropropane-1,3-Diol | 0.02 |
| Mixture of oligofucoses-1 | 0.50 |
| Vitamin A | 3.50 |
| Vitamin C | 1.00 |

| Component | % (by weight) |
|---|---|
| Water | q.s.p. 100% |
| Sodium benzoate | 0.2 |
| Disodium EDTA | 0.08 |
| Glycerin | 2.00 |
| Butylene glycol | 4.00 |
| Carbomer ETD 2020 | 0.20 |
| Ceteareth-20 | 1.00 |
| Mineral oil | 3.00 |
| Squalane | 2.00 |
| Octyl palmitate | 6.00 |
| Karite butter (“Shea butter”) | 2.50 |
| Cetearyl alcohol | 1.00 |
| Rosa AFFF Rubiginosa seed oil | 0.20 |
| Decyl oleate | 0.50 |
| BHA | 0.01 |
| Cyclomethicone | 5.00 |
| Cyclomethicone & Dimethiconol | 2.00 |
| Dimethicone | 2.00 |
| Fragrance (Crematest Feno) | 0.09 |
| Fragrance (Chemoderm) | 0.09 |
| Triethanolamine | 0.30 |
| 2-Bromo-2-Nitropropane-1,3-Diol | 0.02 |
| Mixture of oligofucoses-1 | 0.50 |
| Vitamin A | 3.50 |
| Vitamin C | 1.00 |

1. A cosmetic or pharmaceutical composition characterized in that it comprises at least one vitamin component chosen from the group consisting of vitamin C and its derivatives, vitamin A (or retinol) and its derivatives, and mixtures of these components, in association with at least one fucose component chosen from the group consisting of polysaccharides and oligosaccharides that contain fucose and mixtures of these components, as well as at least cosmetically or pharmaceutically acceptable excipient, with the proviso that the vitamin component is not ascorbyl palmitate nor Vitamin A palmitate.

2. A composition according to claim 1, characterized in that the vitamin component is chosen from the group consisting of ascorbic acid, salts and esters thereof, retinol, retinoids other than retinol, and mixtures of the latter.

3. A composition according to claim 1 or 2, characterized in that the vitamin component is chosen from the group consisting of ascorbic acid, sodium ascorbate, ascorbyl phosphate, retinol, retinoic acid, retinaldehyde, and mixtures of these components.

4. A composition according to any one of the preceding claims, characterized in that the fucose component is chosen from the group consisting of polysaccharides and oligosaccharides comprising the repetition motif fucose-galactose-galacturonic acid, and mixtures of them latter.

5. A composition according to any one of the preceding claims, characterized in that the fucose component is a mixture of non-sulfated fucose-based oligosaccharides, which comprises oligosaccharides of less than 13 saccharide units that comprise at least one fucose unit in a non-reducing end position, and in that it is susceptible of being obtained by means of a process that comprises at least one step of degradation of a polysaccharide from a microorganism of the gender Klebiela pneumoniae subsp. pneumoniae.

6. A composition according to claim 5, characterized in that the mixture of non-sulfated fucose-based oligosaccharides comprises, based on the total weight of the mixture, at least 15% by weight of oligosaccharides of less than 13 saccharide units that comprise at least one fucose unit in a non-reducing end position.

7. A composition according to claim 5 or 6, characterized in that the mixture of non-sulfated fucose-based oligosaccharides comprises, based on the total weight of the mixture, from 20 to 50% by weight of oligosaccharides of less than 13 saccharide units that comprise at least one fucose unit in a non-reducing end position.

8. A composition according to any one of claims 5-7, characterized in that the mixture of non-sulfated fucose-based oligosaccharides comprise, on the other hand, based on the total weight of the mixture, from 25 to 45% by weight of oligosaccharides that have from 13 to 24 saccharide units that comprise at least one fucose unit in a non-reducing end position.

9. A composition according to any one of claims 5-8, characterized in that the mixture of non-sulfated fucose-based oligosaccharides comprise, on the other hand, based on the total weight of the mixture, from 15 to 35% by weight of oligosaccharides of more than 54 saccharide units that comprise at least one fucose unit in a non-reducing end position.

10. A composition according to any one of claims 5-9, characterized in that the non-sulfated fucose-based oligosaccharides comprise, at least in part, the repetition motif fucose-galactose-galacturonic acid.

11. A composition according to any one of claims 5-10, characterized in that the mixture of non-sulfated fucose-based oligosaccharides is susceptible of being obtained by means of a process that comprises the steps of:
a) causing the microorganism of the gender Klebsiella pneumoniae subsp. pneumoniae to grow in an aqueous nutritive medium by aerobic fermentation of an assimilable source of glucide;
b) recovering the polysaccharide formed from the fermentation must;
c) subjecting the polysaccharide to a moderate hydrolysis;
d) subjecting the hydrolysis product of step c) to an enzymatic hydrolysis; and
e) deactivating the enzyme and then recovering the Mixture of oligosaccharides thus formed.

12. A composition according to any one of claims 5-11, characterized in that the microorganism Klebsiella pneumoniae subsp. pneumoniae is the microorganism deposited in the National Collection of Cultures of Microorganisms under Number I-1507, or a mutant thereof.

13. A composition according to claim 11 or 12, characterized in that the moderate hydrolysis for preparing the mixture of non-sulfated fucose-base oligosaccharides is carried out by means of a treatment chosen from the group consisting of treatments with gamma rays, protolysis treatments and combinations of these treatments.

14. A composition according to any one of claims 11-13, characterized in that the enzymatic hydrolysis for preparing the mixture of non-sulfated fucose-based oligosaccharides is carried out with at least one endo-fucosidase.

15. A mixture according to claim 14, characterized ion that the endo-fucosidase is Fermizyme HCP.

16. A composition according to any one of the preceding claims, characterized in that the concentration of the fucose component ranges from about 0.001 to about 20% by weight, and that the concentration in vitamin component ranges from about 0.001 to about 90% by weight, based on the total weight of the composition.

17. A composition according to any one of the preceding claims, characterized in that the weight ratio of vitamin component:fucose component ranges from about 800:1 to about 1:2.

18. A composition according to any one of the preceding claims, characterized in that it comprises, on the other hand, a vector in the form of microspheres that contain the vitamin component.

19. A composition according to any one of the preceding claims, characterized in that it comprises, on the other hand, at least one cosmetically or pharmaceutically acceptable additive chosen from the group consisting of skin-structuring agents, moistening agents, emollient agents, silicones, sun-protection agents, emulsifiers, thickeners, scavengers, antioxidants, fragrances, preservatives, and mixtures thereof.

20. Use, in a cosmetic or pharmaceutical composition, of at least one vitamin component as defined in any one of claims 1-3, in association with at least one fucose component as defined in any one of claims 1 and 4-15, for reducing the toxic effects of the vitamin component.

21. Use according to claim 20, characterized in that the weight ratio of vitamin component:fucose component ranges from about 800:1 to about 1:2.

22. A method of cosmetic treatment of the skin, characterized in that one applies to the skin a cosmetic composition as defined in any one of claims 1-19.
